(19) United States
(12) Patent Application Publication
(10) Pub. No.: US 2023/0310509 A1
(43) Pub. Date: Oct. 5, 2023

(54) Treatment of Alzheimer's Disease with Allogeneic Mesenchymal Stem Cells

(71) Applicant: Longeveron, Inc., Miami, FL (US)

(72) Inventors: Joshua M. HARE, Miami, FL (US); Anthony A. OLIVA, Miami, FL (US)

(73) Assignee: Longeveron, Inc., Miami, FL (US)

(21) Appl. No.: 18/044,092

(22) PCT Filed: Sep. 8, 2021
(86) PCT No.: PCT/US2021/071393

§ 371 Date: Mar. 6, 2023

Related U.S. Application Data
(60) Provisional application No. 63/075,686, filed on Sep. 8, 2020.
(60) Provisional application No. 63/134,535, filed on Jan. 6, 2021.
(60) Provisional application No. 63/173,960, filed on Apr. 12, 2021.

(51) Int. Cl. A61K 35/28; A61P 25/28; G01N 33/68

(52) U.S. Cl. CPC A61K 35/28; A61P 25/28; G01N 33/6896; G01N 2800/2821; G01N 2333/54

(57) ABSTRACT

Compositions and methods are disclosed herein for the treatment of Alzheimer's disease with allogeneic mesenchymal stem cells. The methods of treatment involve the administration of a composition of allogeneic mesenchymal stem cells to a subject in need thereof, wherein the effectiveness of the treatment methods can be determined through the measurement of specific biomarkers and improved cognitive function.

### CROSS-REFERENCE TO RELATED APPLICATIONS

This application is related to and claims the benefit of U.S. Provisional Application No. 63/075,686 “TREATMENT OF ALZHEIMER'S DISEASE WITH ALLOGENEIC MESENCHYMAL STEM CELLS”, filed on Sep. 8, 2020, U.S. Provisional Application No. 63/134,535 “TREATMENT OF ALZHEIMER'S DISEASE WITH ALLOGENEIC MESENCHYMAL STEM CELLS”, filed on Jan. 6, 2021 and U.S. Provisional Application No. 63/173,960 “TREATMENT OF ALZHEIMER'S DISEASE WITH ALLOGENEIC MESENCHYMAL STEM CELLS”, filed on Apr. 12, 2021, the entire contents of each being incorporated herein by reference in their entirety.

### FIELD

The present application relates to methods and compositions for the treatment of Alzheimer's disease in subjects in need thereof. Some embodiments are drawn to compositions comprising a therapeutically effective amount of allogeneic mesenchymal stem cells (MSCs), which are used to alleviate the symptoms of Alzheimer's disease, such as increased systemic inflammation. Other embodiments are drawn to methods of treatment wherein subjects suffering from symptoms of Alzheimer's disease are administered compositions comprising a therapeutically effective amount of MSCs. The effectiveness of these treatments is evaluated through measuring the concentrations of specific biomarkers in subjects after administration of compositions comprising MSCs, examining changes in their brain activity or morphology and determining if their cognitive functioning has improved after treatment.

### BACKGROUND

Alzheimer's disease (AD) involves complex pathology and encompassing diverse mechanisms in addition to β-amyloid deposition and neurofibrillary tangles [1]. There is growing recognition that a pro-inflammatory state contributes to the ensuing dementia [2-4]. In this regard, proinflammatory cytokines are abundant in the vicinity of amyloid deposits and neurofibrillary tangles [5], and an association exists between systemic inflammation and β-amyloid accumulation [4]. AD is further characterized by impaired neurovasculature that contributes to adverse outcomes [6]. Resulting compromise of the blood-brain barrier (BBB) [7-10] can impair exchange across the endothelium leading to inefficient clearance and accumulation of AβP in the brain [11, 12].

Due to the complex nature of AD progression, the use of biomarkers to predict AD onset and progression remains challenging. Though the concentration of β-amyloid deposits and neurofibrillary tangles can be used to diagnose or predict the onset of AD, there are individuals that have been shown to possess a significant amount of amyloid deposits and neurofibrillary tangles at autopsy, which would qualify them for a diagnosis of AD, which never showed a history of dementia. Currently approved treatments for AD (Rivastigmine, Donepezil, Memantine, Galantamine, Tacrine) have only marginal benefits that are largely symptomatic; and no approved therapies can effectively stop, reverse, or prevent AD. The consistent failure of initially-promising lead compounds have resulted in no new AD drugs approved in over a decade. Recent among these are the failures of the anti-amyloid monoclonal antibody, solanezumab (Ely Lily) and aducanumab (Biogen/Eisai), which were found ineffective in mild or moderate stage AD, and mild cognitive impairment (MCI). A common theme among these failures is the targeting of a single pathological feature of AD.

Addressing these neuropathological features of AD simultaneously could offer therapeutic advantages and lead to novel treatment strategies. Medicinal signaling cells (MSCs, also known as mesenchymal stem cells) are multipotent cells (in vitro) with pleiotropic mechanisms of action (MOAs), including anti-inflammatory properties, ability to improve vascular function, and promotion of intrinsic tissue repair and regeneration [13, 14]. MSCs traffic to sites of inflammation and damage, and thus could target sites of neuroinflammation in AD. MSCs can also regulate host stem cell niches through paracrine activity and heterocellular coupling to promote intrinsic repair and regeneration [15]. Finally, MSCs are immunoevasive/immunoprivileged, permitting allogeneic use, and have an acceptable safety profile in clinical trials. These immunoprivileged/immunoevasive properties allow MSCs to have the potential to be an “off-the-shelf” therapy that is readily available and accessible to broad patient populations due to their undetectable levels of major histocompatibility complex class II (MHC-II) molecules and low levels of MHC-I.

There are some preclinical data supporting efficacy of MSCs in AD. In animal models, MSCs cross the BBB, promote neurogenesis, inhibit β-amyloid deposition and promote clearance, reduce apoptosis, promote hippocampal neurogenesis, improve dendritic morphology, and improve behavioral and spatial memory performance [18-20]. These beneficial effects were associated with decreased inflammation, increased Aβ-degrading factors and Aβ clearance, decreased hyperphosphorylated tau, and elevated alternatively activated microglial markers. These benefits appear, at least in part, due to Aβ-induced MSC release of chemoattractants that recruit alternative microglia into the brain to reduce Aβ deposition [21]. MSCs have been reported to be effective in young AD-model mice prior to Aβ accumulations, leading to significant decreases in cerebral Aβ deposition, and a significant increase in expression of pre-synaptic proteins [22]. Impressively, these effects were sustained for at least 2 months, and suggest MSCs could potentially be effective as an interventional therapeutic in prodromal AD.

Accordingly, the application seeks to not only provide methods of treatment for AD wherein the methods comprise the use of compositions containing MSCs, but this application also seeks to provide methods that can accurately measure the potential safety of MSCs and evaluate their efficacy in the alleviation of AD symptoms in subjects in need thereof.

### SUMMARY

An objective of the present application is to provide methods of treatment or alleviation for AD that comprise administering a therapeutic amount of allogeneic MSCs to a subject in need thereof to alleviate the symptoms and/or treat the progression of AD. Another objective of the present application is to provide novel biomarkers for diagnosing and evaluating the progression of AD and the effectiveness of the treatment methods. These biomarkers may be the change in the size in areas of a patient's brain, such as the amygdala, cortical nucleus, the hippocampus, hippocampal subregions, and/or the corticoamygdaloid transition.

In some embodiments, the novel biomarkers for diagnosing and evaluating the progression of AD may be a change in a cytokine's concentration, wherein the cytokine may be IL-4, IL-6, IL-8, IL-10, IL-12p70, IL-17, sIL-2Rα or combinations thereof. In preferred embodiments, the cytokine's concentration is increased in the serum, plasma, cerebral spinal fluid or blood of the subject in need thereof suffering from AD symptoms after administration of allogenic MSCs to said subject. The cytokine concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the cytokine concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

In other embodiments, the novel biomarkers for diagnosing and evaluating the progression of AD may be a change in a neuronal-related molecule or peptide's concentration, wherein the neuronal signaling molecule or peptide may be tau, phospho-tau, Aβ-38, Aβ-40, Aβ-42, NFL or combinations thereof. In preferred embodiments, the concentration of Aβ-38, Aβ-40 or Aβ-42 is increased in the serum, plasma or blood of the subject in need thereof suffering from AD symptoms after administration of allogenic MSCs to said subject. The Aβ-38, Aβ-40 or Aβ-42 concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the Aβ-38, Aβ-40 or Aβ-42 concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

In other embodiments, the concentration of tau, phospho-tau, or NFL is decreased in the serum, plasma, cerebral spinal fluid or blood of the subject in need thereof suffering from AD symptoms after administration of allogenic MSCs to said subject. The tau, phospho-tau, or NFL concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the tau, phospho-tau, or NFL concentration is decreased to a stable concentration level wherein the concentration does not increase more than 0% to 10%, 0% to 5% or 0% to 1% once it has reached and maintained a concentration level that is different from the concentration level before administration of MSCs to the subject in need thereof.

In other embodiments, the novel biomarkers for diagnosing and evaluating the progression of AD may be a change in an inflammation signaling molecule's concentration, wherein the inflammation signaling molecule may be pro-BNP, TNF-α, or combinations thereof. In preferred embodiments, the concentration of TNF-α or pro-BNP is decreased in the serum, plasma, cerebral spinal fluid or blood of the subject in need thereof suffering from AD symptoms after administration of allogenic MSCs to said subject. The TNF-α or pro-BNP concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the TNF-α or pro-BNP concentration is decreased to a stable concentration level wherein the concentration does not increase more than 0% to 10%, 0% to 5% or 0% to 1% once it has reached and maintained a concentration level that is different from the concentration level before administration of MSCs to the subject in need thereof.

In some embodiments, the novel biomarkers for diagnosing and evaluating the progression of AD may be a change in VEGF concentration and other vascular-related biomarkers. In preferred embodiments, the concentration of VEGF is increased in the serum, plasma, cerebral spinal fluid or blood of the subject in need thereof suffering from AD symptoms after administration of allogenic MSCs to said subject. The VEGF concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the VEGF concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

The allogenic MSCs may be LOMECEL-B™ cells, which is a Longeveron formulation of allogenic human mesenchymal stem cells. Further uses and preparation of useful stem cells, including LOMECEL-B™ brand mesenchymal cells, may be found in the following United States patent application Publications, all of which are incorporated by reference herein: US20190038742A1; US20190290698A1; and US20200129558A1.

### BRIEF DESCRIPTION OF DRAWINGS

FIG. 1 illustrates a Phase 1 double-blinded, randomized, and placebo-controlled clinical trial that was performed to determine the effectiveness of Lomecel-B in treating subjects diagnosed with mild AD.

FIG. 2 depicts the experimental groups used in the Phase 1 clinical trial and how the subjects were divided into each experimental group.

FIG. 3A depicts the MMSE scores of the three experimental groups (20×10⁶ Lomecel-B, 100×10⁶ Lomecel-B and placebo) over a six month period. FIG. 3B depicts the changes in ADAS-cog scores of the three experimental groups (20×10⁶ Lomecel-B, 100×10⁶ Lomecel-B and placebo) over a six month period. FIG. 3C depicts the changes in TMT-A scores of the three experimental groups (20×10⁶ Lomecel-B, 100×10⁶ Lomecel-B and placebo) over a six month period. FIG. 3D depicts the changes in TMT-B scores of the three experimental groups (20×10⁶ Lomecel-B, 100×10⁶ Lomecel-B and placebo) over a six month period. FIG. 3E depicts the changes in GDS points of the three experimental groups (20×10⁶ Lomecel-B, 100×10⁶ Lomecel-B and placebo) over a six month period.

FIG. 4A depicts the changes in the subject version of the QOL-AD points of the three experimental groups (20×10⁶ Lomecel-B, 100×10⁶ Lomecel-B and placebo) over a six month period. FIG. 4B depicts the changes in the caregiver version of the QOL-AD points of the three experimental groups (20×10⁶ Lomecel-B, 100×10⁶ Lomecel-B and placebo) over a six month period. FIG. 4C depicts the changes in the ADCS-ADL points of the three experimental groups (20×10⁶ Lomecel-B, 100×10⁶ Lomecel-B and placebo) over a six month period. FIG. 4D depicts the changes in the ADRQL points of the three experimental groups (20×10⁶ Lomecel-B, 100×10⁶ Lomecel-B and placebo) over a six month period.

FIG. 5A depicts the relative VEGF concentration change in the serum of the three experimental groups (20×10⁶ Lomecel-B, 100×10⁶ Lomecel-B and placebo) over a six month period. FIG. 5B depicts the relative IL-4 concentration change in the serum of the three experimental groups (20×10⁶ Lomecel-B, 100×10⁶ Lomecel-B and placebo) over a six month period. FIG. 5C depicts the relative IL-6 concentration change in the serum of the three experimental groups (20×10⁶ Lomecel-B, 100×10⁶ Lomecel-B and placebo) over a six month period. FIG. 5D depicts the relative sIL-2Rα concentration change in the serum of the three experimental groups (20×10⁶ Lomecel-B, 100×10⁶ Lomecel-B and placebo) over a six month period. FIG. 5E depicts the relative IL-10 concentration change in the serum of the three experimental groups (20×10⁶ Lomecel-B, 100×10⁶ Lomecel-B and placebo) over a six month period. FIG. 5F depicts the relative IL-12 concentration change in the serum of the three experimental groups (20×10⁶ Lomecel-B, 100×10⁶ Lomecel-B and placebo) over a six month period.

FIG. 6A depicts the brain volumetry changes in the left hippocampal region of the three experimental groups (20×10⁶ Lomecel-B, 100×10⁶ Lomecel-B and placebo) over a six month period. FIG. 6B depicts the brain volumetry changes in the right hippocampal region of the three experimental groups (20×10⁶ Lomecel-B, 100×10⁶ Lomecel-B and placebo) over a six month period.

### DETAILED DESCRIPTION

There is only 1 FDA-approved disease-modifying intervention for AD (Aducanumab), which is controversial, and may possibly delay the progression of dementia in a subpopulation of AD patients. The other FDA-approved treatments for AD are only symptomatic treatments, and do not alter disease progression. The development and onset of AD is still not fully understood and this uncertainty has been the cause of many disputes within the field. Indeed, though a majority of researchers in the field of AD development and treatment acknowledge that AβP accumulation plays a part in the progression of the disease, it is still unknown if AβP accumulation is the cause of AD or if it is merely a result of other cellular pathways becoming dysregulated as a result of aging.

Patients suffering from AD have been known to elicit irregular immune responses. Indeed, it has been shown that an abundance of pro-inflammatory cytokines exist in the vicinity of amyloid deposits and neurofibrillary tangles, thus hinting at an association between systemic inflammation and β-amyloid accumulation. Even in light of this evidence, those skilled in the art have repeatedly been skeptical of the immune system's role in the development of AD since there has been no direct correlation between the inhibition of pro-inflammatory cytokines in humans and the reduction of AβP accumulation.

Accordingly, we have surprisingly discovered that the use of a composition comprising allogeneic MSCs is able to combat the symptoms of AD. Treating a subject suffering from AD symptoms with a composition comprising allogeneic stem cells has been discovered to improve the subject's brain morphology and promote the expression of biomarkers that are associated with anti-inflammation and vascular repair. We have also discovered that allogeneic MSCs are capable of promoting improvements in neuroinflammation and vascular function of a subject suffering from symptoms of AD. The above discoveries are surprising due to the ambiguity surrounding the pathogenesis of AD and the general reservation of those skilled in the art to use MSCs in treatments for AD since they were expected to perform poorly due to their inability to directly target beta-amyloids and their low residence time in the human body. They were also expected to perform poorly in AD treatments due to their large size, which led those skilled in the art to believe that they could not pass the blood-brain barrier and reach the site of inflammation and damage.

Another advantage of using MSCs in treatments for AD is that they do not involve targeting a single pathway or biomarker, such as AβP accumulation. Instead, the use of MSCs in AD treatments can allow multiple pathways to be targeted at once and thereby halt or significantly slow the progression of AD.

Following the surprising discoveries discussed above, one aspect of the present application relates to methods of treating AD or alleviating the symptoms of AD, wherein the methods comprise administering to a subject suffering from symptoms of AD a composition comprising allogenic MSCs.

In some embodiments, the method of treatment for AD or alleviating the symptoms of AD further comprises measuring the concentration of biomarkers in the subject suffering from symptoms of AD before and/or after the administration of the composition comprising allogenic MSCs.

In other embodiments, the method of treatment for AD or alleviating the symptoms of AD further comprises measuring the subject suffering from symptoms of AD cognitive function before and/or after administration of the composition comprising allogenic MSCs.

In some embodiments, the MSCs used in the methods of treatment are Lomecel-B™ MSCs.

In other embodiments, the biomarkers are cytokines such as IL-4, IL-6, IL-8, IL-10, IL-12p70, IL-17, sIL-2Rα or combinations thereof.

In preferred embodiments, the cytokine's concentration is increased in the serum, plasma, cerebral spinal fluid or blood of the subject in need thereof suffering from AD symptoms after administration of allogenic MSCs to said subject. The cytokine concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the cytokine concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

In other embodiments, the biomarkers are neuronal-related molecules or peptides such as tau, phospho-tau, Aβ-38, Aβ-40, Aβ-42, NFL or combinations thereof.

In preferred embodiments, the concentration of Aβ-38, Aβ-40 or Aβ-42 is increased in the serum, plasma or blood of the subject in need thereof suffering from AD symptoms after administration of allogenic MSCs to said subject. The Aβ-38, Aβ-40 or Aβ-42 concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other embodiments, the Aβ-38, Aβ-40 or Aβ-42 concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

In other embodiments, the concentration of tau, phospho-tau, or NFL is decreased in the serum, plasma, cerebral spinal fluid or blood of the subject in need thereof suffering from AD symptoms after administration of allogenic MSCs to said subject. The tau, phospho-tau, or NFL concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other embodiments, the tau, phospho-tau, or NFL concentration is decreased to a stable concentration level wherein the concentration does not increase more than 0% to 10%, 0% to 5% or 0% to 1% once it has reached and maintained a concentration level that is different from the concentration level before administration of MSCs to the subject in need thereof.

In other embodiments, the biomarkers are inflammation signaling molecules such as pro-BNP, TNF-α, or combinations thereof.

In preferred embodiments, the concentration of TNF-α or pro-BNP is decreased in the serum, plasma, cerebral spinal fluid or blood of the subject in need thereof suffering from AD symptoms after administration of allogenic MSCs to said subject. The TNF-α or pro-BNP concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In other embodiments, the TNF-α or pro-BNP concentration is decreased to a stable concentration level wherein the concentration does not increase more than 0% to 10%, 0% to 5% or 0% to 1% once it has reached and maintained a concentration level that is different from the concentration level before administration of MSCs to the subject in need thereof.

In some embodiments, the biomarker is VEGF or other vascular-related biomarkers.

In preferred embodiments, the concentration of VEGF is increased in the serum, plasma, cerebral spinal fluid or blood of the subject in need thereof suffering from AD symptoms after administration of allogenic MSCs to said subject. The VEGF concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the VEGF concentration is increased to a stable concentration level wherein the concentration does not decline more than 0% to 10%, 0% to 5% or 0% to 1% once it has reached and maintained a concentration level that is different from the concentration level before administration of MSCs to the subject in need thereof.

In other embodiments, the method of treatment for AD or alleviating the symptoms of AD further comprises determining the change in the size of areas in the subject's brain after administration of the compositions comprising allogeneic MSCs. The areas in the subject's brain that can change in size may be the amygdala, cortical nucleus, hippocampus, or other structures.

In other embodiments, the method of treatment for AD or alleviating the symptoms of AD further comprises examining the cerebral spinal fluid of a subject before and after administration of the compositions comprising allogeneic MSCs.

In other embodiments, the method of treatment for AD or alleviating the symptoms of AD further comprises examining the blood serum of a subject before and after administration of the compositions comprising allogeneic MSCs.

In other embodiments, the method of treatment for AD or alleviating the symptoms of AD further comprises examining the blood plasma of a subject before and after administration of the compositions comprising allogeneic MSCs.

In some embodiments, the method of treatment for AD or alleviating the symptoms of AD further comprises determining if a change in the corticoamygdaloid transition of the subject has occurred after administration of the composition comprising allogeneic HMCs.

In other embodiments, the composition may contain either 20×10⁶ MSCs, 100×10⁶ MSCs or between 20×10⁶ and 100×10⁶ MSCs.

### EXAMPLES

### Example 1: Double-Blind Phase I Clinical Trail Evaluating the Effectiveness of MSCs in the Treatment of AD Symptoms

### Trial Design:

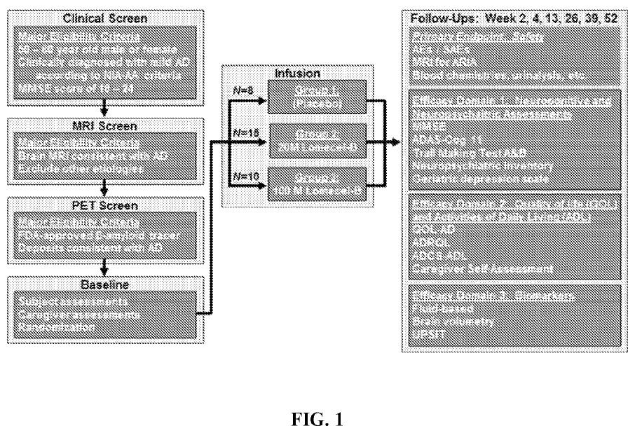

The Phase 1 trial was double-blinded, randomized, and placebo-controlled (FIG. 1), was registered with ClinicalTrials.gov (NCT02600130), and was under oversight by a single Institutional Review Board, independent Data and Safety Monitoring Board (DSMB), independent clinical monitors, and Food and Drug Administration (FDA) under an Investigation New Drug Application (IND). All subjects and caregivers were consented to participate on the trial. Subject screening consisted of a 3-tiered process consisting of clinical assessment for probable mild AD, an MRI to exclude confounding issues, and an amyloid tracer PET scan to confirm the mild AD diagnosis. Enrolled subjects were randomized to receive a single infusion of low-dose of Lomecel-B [2.0×10⁷ cell (“20M”)], high-dose of Lomecel-B [(1.0×10⁸ cells (“100M”)], or placebo. As enrollment neared completion, all subjects in screening were enrolled if they met eligibility, resulting in 33 subjects enrolled (versus the projected 30). The infusion day was defined as Day 0. Follow-ups were at Weeks 2, 4, 13, 26, 39, and 52 post-infusion.

### Lomecel-B and Placebo:

Lomecel-B is a formulation of allogeneic MSCs sourced from healthy young adult donors in compliance with the Codes of Federal Regulations 1271, and culture-expanded using current Good Manufacturing Practices (cGMP) under and an FDA-approved Chemistry, Manufacturing, and Controls (CMC) section of an IND. The placebo consisted of vehicle that Lomecel-B MSCs are resuspended in (PlasmaLyte-A with 1% human serum albumin). Lomecel-B and placebo were prepared in identically-appearing infusion bags bearing identical appearing labels, and delivered via peripheral intravenous infusion in an out-patient setting.

### Clinical Assessments:

Clinical assessments were performed at baseline, and Week 2, 13, 26, 39, and 52, except for the MMSE [24], which was at the screening visit (an enrollment criterion) in place of the baseline visit. Clinical assessments used were the 11-part Alzheimer's Disease Assessment Scale—Cognitive subscale (ADAS-Cog), Trail Making Test parts A & B (TMT-A and TMT-B), Neuropsychiatric Inventory (NPI), short version of the Geriatric Depression Scale (GDS), ADCS-ADL, Alzheimer's Disease Related Quality of Life (ADRQL), American Medical Association-developed Caregiver Self-Assessment Questionnaire, and patient and caregiver versions of the QOL-AD.

### Biomarkers:

Assays were performed by a central laboratory (Cenetron Diagnostics: Austin, TX) for Vascular Endothelial Growth Factor (VEGF), D-dimer, N-Terminal ProB-type Natriuretic Peptide, Transforming growth factor-β1, C-reactive protein, Interleukin- (IL-) 5, IL-17, and soluble IL-2Rα (sIL-2Rα). High-sensitivity electrochemiluminescence immunoassays were performed by Longeveron using the MESO QuickPlex SQ 120 system (Meso Scale Diagnostics, LLC: Rockville, MD) for IL-1β, IL-2, IL-4, IL-6, IL-8, IL-10, IL-12p70, IL-13, tumor necrosis factor-α (TNF-α), TNF-α stimulated gene 6, interferon-gamma, amyloid beta (Aβ) peptide 1-38 (Aβ₃₈), Aβ₄₀, Aβ₄₂, total Tau, phospho-Tau T181, and neurofilament light chain (NfL). CSF collection was made optional in this safety trial, and limited samples precluded formal statistical analyses.

Brain MRI was performed at screening, and Weeks 13, 26, 39, and 52 to assess for safety (including ARIA), and further used for evaluating structural brain changes.

The screening PET used florbetaben (18^{F}) (Life Molecular Imaging: Boston, MA). Patients with a positive amyloid tracer PET scan prior to screening were allowed to enroll without requiring this scan (provided they met enrollment criteria).

### Statistical Analysis:

Sample size was chosen to yield a 79% probability of detecting Adverse Events (AEs) that occur at a rate of 5% or more. Analyses was performed by an independent third-party statistician groups. Two-sided test were used to perform statistical tests to 0.05 significance level, and 95% confidence intervals calculated when appropriate. Adjustments for multiple analyses were not performed.

The primary endpoint was triggering of the Bayesian motivated safety stopping rule for incidence of Serious Adverse Events (SAEs) within the first 30 days post-infusion (defined as Treatment-Emergent Serious Adverse Events, or TE-SAEs). Boundaries were calculated based on an assumed TE-SAE rate of 10.0%, and a TE-SAE rate>40% would trigger the stopping rule. The stopping rule had a 19% chance of Type I error, and was 91% powered.

Additional safety assessments included the following, AEs and SAEs were evaluated throughout the study. Clinical laboratory testing (hematology, blood chemistry, coagulation, and urinalysis) was performed at the screening, baseline, and infusion visits, and Weeks 4, 13, 26, 39, and 52. Physical and neurological examinations were also performed. Electrocardiogram (ECG) was performed at the screening and infusion visits, and Weeks 4 and 52.

Overall follow-up compliance was 100% through Week 13 post-infusion, and 85% through Week 26 [13 out of 15 (87%) for the low-dose Lomecel-B arm, 8 out of 10 (80%) for the high-dose Lomecel-B, and 7 out of 8 (88%) for the placebo]. Thereafter, follow-up compliance dropped such that 5 patients (33%) for the low-dose Lomecel-B arm, 6 patients (60%) for high-dose Lomecel-B, and 2 patients (13%) for placebo, were withdrawn before the 52 Week follow-up visit (61% overall compliance at Week 52). Six of the 13 withdrawals (46%) were during the COVID-19 pandemic. As such, efficacy is only presented through Week 26.

### Study Population:

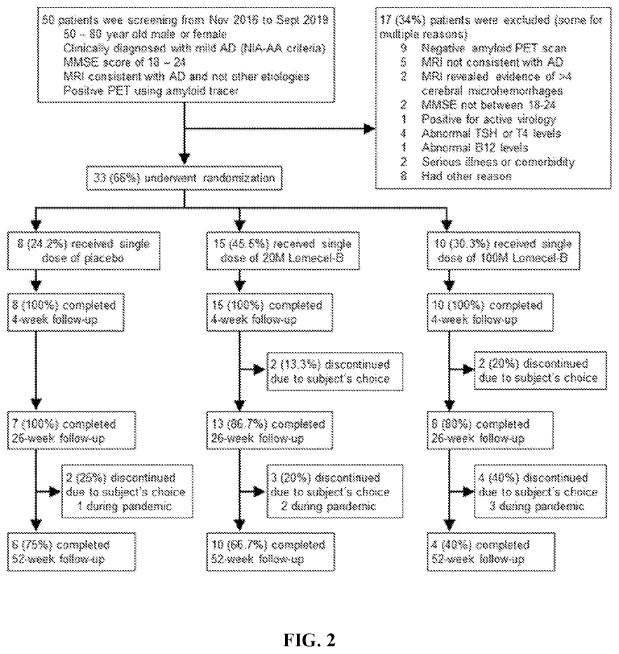

Fifty subjects were screened at 4 clinical sites, and 33 (66%) were enrolled and randomized between Nov. 3, 2016 and Sep. 19, 2019 (FIG. 2). The two main reasons for screen failure were a negative amyloid-tracer PET scan (52% of screen fails) or concurrent MRI findings (29% of screen fails). Enrolled subjects received a single intravenous infusion of 20M Lomecel-B (N=15), 100M Lomecel-B (N=10), or placebo (N=8).

Baseline demographics are presented in Table 1. The mean age was 71.2±8.4 years, and 48.5% were female. At least 19 of the subjects (57.6%) carried at least one ApoE4 allele (2 subjects declined genetic testing).

TABLE 1
Baseline Demographics

| | Placebo | | 20M Lomecel-B | | 100M Lomecel-B | |
|---|---|---|---|---|---|---|
| Variable | (n = 8) | | (n = 15) | | (n = 10) | |
| Age (Years) Mean + SD | 75.9 ± 5.03 | | 70.1 ± 9.49 | | 69.3 ± 8.08 | |
| Female sex [n (%)] | 6 | (75.0) | 4 | (26.7) | 6 | (60.0) |

Ethnicity and Race [n (%)]

| | | | | | | |
|---|---|---|---|---|---|---|
| Hispanic or Latino | 1 | (12.5) | 3 | (20) | 2 | (20.0) |
| Not Hispanic or Latino | 7 | (87.5) | 12 | (80) | 8 | (80.0) |
| White | 6 | (75) | 13 | (86.7) | 10 | (100.0) |

| | | | | | |
|---|---|---|---|---|---|
| Black/African American | 2 | (29) | 1 | (6.7) | 0 |

| | | | | |
|---|---|---|---|---|
| More than One Race | 0 | 1 | (6.7) | 0 |

APOE Genotype [n (%)]

| e2/e3 | 2 | (25.0) | 0 | | 0 | |
|---|---|---|---|---|---|---|
| e3/e3 | 1 | (12.5) | 6 | (40.0) | 3 | (30.0) |
| e3/e4 | 4 | (50.0) | 7 | (46.7) | 5 | (50.0) |
| e4/e4 | 1 | (12.5) | 1 | (6.65) | 1 | (10.0) |

| | | | | | |
|---|---|---|---|---|---|
| Unknown | 0 | 1 | (6.65) | 1 | (10.0) |

Clinical assessment (Points)
Mean ± SD (range)

| | | | |
|---|---|---|---|
| Mini Mental State Exam | 20.45 ± 1.46 | 20.60 ± 2.06 | 20.70 ± 2.26 |
| | (18.0-22.0) | (18.0-23.0) | (18.0-24.0) |
| ADAS-cog-11 | 23.46 ± 6.34 | 24.71 ± 8.49 | 25.07 ± 8.30 |
| | (15.7-37.7) | (12.7-43.3) | (12.7-38.7) |
| ADCS-ADL | 57.60 ± 11.16 | 58.93 ± 13.33 | 50.40 ± 19.87 |
| | (44.0-74.0) | (31.0-73.0) | (20.0-73.0) |
| ADRQL | 78.3 ± 17.5 | 89.1 ± 10.7 | 82.2 ± 16.8 |
| | (46.3-98.0) | (69.0-100.0) | (42.0-97.5) |
| GDS | 2.80 ± 2.600 | 1.10 ± 1.410 | 1.50 ± 1.270 |
| | (0-7) | (0-4) | (0-4) |
| NPI | 36.6 ± 39.7 | 15.1 ± 14.2 | 28.3 ± 27.2 |
| | (1-125) | (1-46) | (2-94) |
| QOL-AD (patient version) | 36.3 ± 7.3 | 37.4 ± 4.8 | 37.5 ± 4.9 |
| | (25-44) | (30-46) | (30-45) |
| QOL-AD (caregiver version) | 28.80 ± 4.59 | 32.50 ± 6.85 | 31.00 ± 7.02 |
| | (24-37) | (20-42) | (22-43) |
| Trail Making Test Part A | 119.4 ± 64.0 | 101.6 ± 78.2 | 189.8 ± 111.3 |
| | (46-232) | (28-300) | (46-300) |
| Trail Making Test Part B | 263.1 ± 104.3 | 260.0 ± 64.1 | 279.6 ± 61.8 |
| | (5-300) | (100-300) | (104-300) |

Plasma Biomarkers
Mean ± SD (range)

| IL-4 (pg/mL) | 0.08 ± 0.04 | 0.13 ± 0.10 | | 0.10 ± 0.06 | |
|---|---|---|---|---|---|
| | (0.04-0.12) | (0.04-0.34) | | (0.04-0.23) | |
| IL-6 (pg/mL) | 4.52 ± 8.21 | 1.94 ± 1.85 | | 1.68 ± 1.15 | |
| | (0.76-24.79) | (0.71-6.98) | | (0.82-4.80) | |
| IL-10 (pg/mL) | 0.73 ± 0.89 | 0.51 ± 0.23 | | 0.46 ± 0.20 | |
| | (0.19-2.90) | (0.15-1.16) | | (0.19-0.91) | |
| SIL-2Rα (pg/mL) | 589.4 ± 284.9 | 536.1 | (466.1 | 407.7 | (247.5 |

| | | | |
|---|---|---|---|
| | (268.0, 972.0) | (9.0, 1809.0) | (28.0, 774.0) |
| D-Dimer (pg/mL) | 1.97 ± 2.25 | 0.55 ± 0.41 | 0.46 ± 0.32 |
| | (0.29-5.75) | (0.27-1.65) | (0.27-1.06) |
| VEGF (pg/mL) | 52.1 ± 20.3 | 42.8 ± 28.6 | 60.9 ± 39.1 |
| | (32-86) | (11-126) | (15-129) |
| Aβ₃₈ (pg/mL) | 143.9 ± 228.7 | 37573.2 ± 136662.1 | 40.1 ± 28.6 |
| | (26.6-630.2) | (26.6-531361.8) | (26.6-95.5) |
| Aβ₄₀ (pg/mL) | 65.1 ± 60.4 | 2457.1 ± 7676.0 | 82.2 ± 47.8 |
| | (12.1-189.2) | (21.0-29952.8) | (26.6-159.5) |
| Aβ₄₂ (pg/mL) | 12.9 ± 11.2 | 1061.4 ± 3520.2 | 11.5 ± 7.2 |
| | (5.2-39.7) | (7.9-13756.2) | (5.2-28.8) |
| NfL (pg/mL) | 91.8 ± 24.2 | 101.0 ± 58.3 | 79.9 ± 33.3 |
| | (73.0-147.0) | (26.2-270.0) | (40.0-134.9) |
| Total tau (pg/mL) | 44.6 ± 42.3 | 48268.4 ± 174172.4 | 94135.0 ± 278841.3 |
| | (16.5-124.7) | (16.5-677236.6) | (16.5-837665.9) |

### Primary Endpoint—Safety:

The primary endpoint was triggering of the TE-SAE stopping rule. The stopping rule was never triggered, meeting the primary safety endpoint. Only 1 TE-SAE occurred, which was at 27 days post-infusion in the 100M Lomecel-B arm (Table 2) for back pain resulting in 24-hr hospitalization, which was deemed unrelated to study product. The incidence of AEs within 30-days post-infusion (treatment-emergent AEs, i.e., TE-AEs) in the Lomecel-B arms were not different from the placebo arm (16.0% of subjects in the combined Lomecel-B arms, versus 25.0% of subjects in the placebo arm, p<0.1606).

TABLE 2
Incidence of Adverse Events (AEs) and Serious Adverse Events (SAEs)

| | | 20M | 100M |
|---|---|---|---|
| Primary Endpoint | Placebo | Lomecel-B | Lomecel-B |
| Number of SAEs occurring within 30 days after treatment | 0 | 0 | 1 |
| (TE-SAEs) (n) | | | |
| Number of subjects with at least one TE-SAE [n (%)] | 0 | 0 | 1 |
| | | | (10.0%) |
| Number of SAEs occurring over entire trial (n) | 4 | 2 | 3 |
| Number of subjects with at least one SAE [n (%)] | 3 | 2 | 2 |
| | (37.5%) | (13.3%) | (20.0%) |
| Number of AEs occurring within 30 days after treatment | 3 | 3 | 2 |
| (TE-AEs) (n) | | | |
| Number of subjects with at least one TE-AE [n (%)] | 2 | 3 | 1 |
| | (25.0%) | (20.0%) | (10.0%) |
| Number of AEs occurring over entire trial (n) | 33 | 23 | 15 |
| Number of subjects with at least one AE [n (%)] | 7 | 10 | 5 |
| | (87.5%) | (66.7%) | (50.0%) |
| Number of deaths on study (n) | 0 | 1 * | 0 |
| Number of AEs related to study drug (n) | 0 | 0 | 0 |
| Number of SAEs related to study drug (n) | 0 | 0 | 0 |
| Number of infusions interrupted or stopped prematurely (n) | 0 | 0 | 0 |
| Number of patients with ARIA (n) | 0 | 0 | 0 |

* The patient withdrew from the trial first and subsequently died in an assisted-living facility at day 144 after the infusion.

No AEs or SAEs were deemed related to study product. The incidence of SAEs on trial was lower in each Lomecel-B treatment arm versus the placebo arm (16.0% of subjects in the combined Lomecel-B arms, versus 37.5% of subjects in the placebo arm). However, three of these SAEs occurred prior to infusion (all in the placebo arm). There was 1 death on study, which occurred at day 144 post-infusion in the 100M Lomecel-B arm. The incidence of AEs was lower in the Lomecel-B arms versus placebo (60.0% of subjects in the combined Lomecel-B arms, versus 87.0% of subjects in the placebo arm). Only one subject experienced a severe AE (back-pain), which was in the high-dose Lomecel-B arm.

No infusions were interrupted, stopped prematurely, or had an associated AE or SAE. There were no reported incidents of ARIA as assessed by MRI. Hematology, coagulation, blood chemistry, vital signs, urinalysis and ECG data were evaluated by the independent pharmacovigilance monitor and the DSMB, and no trends or causes of concern were observed.

### Neurocognitive and Neuropsychiatric Assessments:

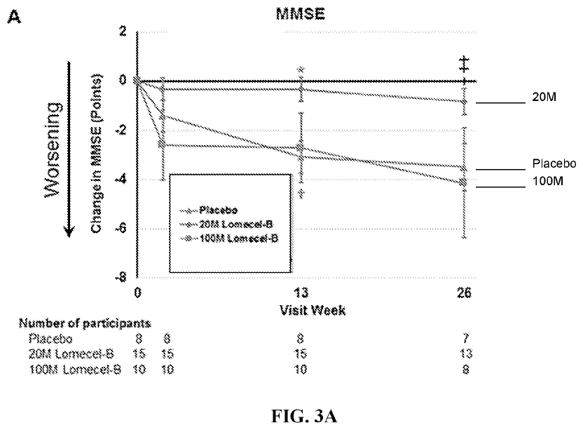

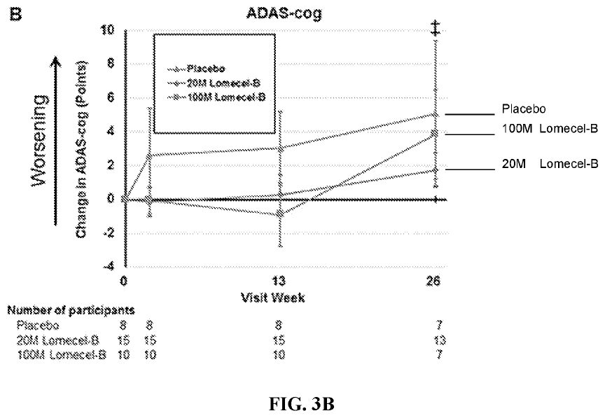

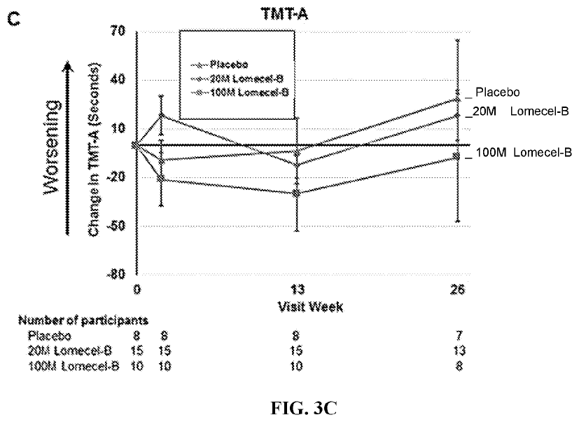

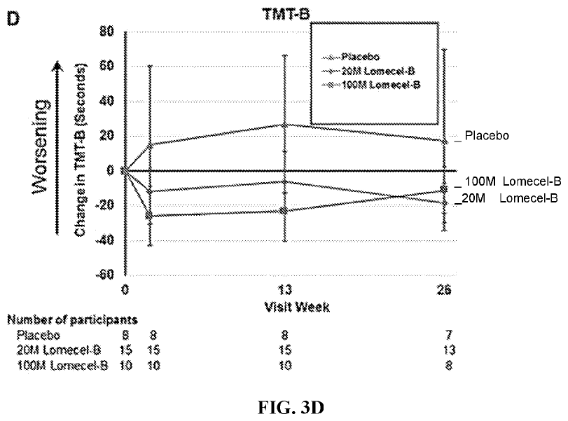

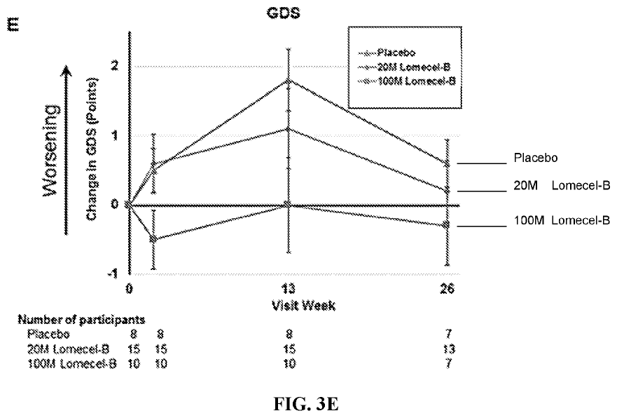

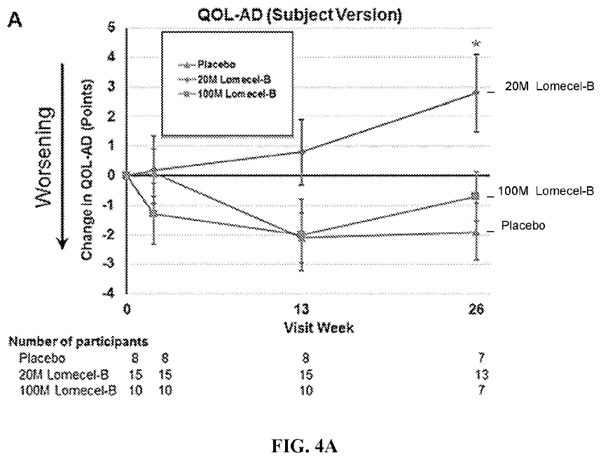

Neurocognitive and neuropsychiatric assessments were evaluated as pre-specified secondary endpoints. In the 20M Lomecel-B arm, the decline in MMSE was significantly slower versus placebo (FIG. 3A). The placebo arm score declined by 2.99±1.12 points at Week 13 (p=0.0337; 2-sided 95% CI −5.84-−0.31). In contrast, the 20M Lomecel-B arm showed no significant change from baseline, and the difference from placebo was significantly higher (better) at Week 13 by 2.69±1.39 points (p=0.0182; 2-sided 95% CI 0.51-4.97). The 100M Lomecel-B arm showed a trending decline in MMSE that did not reach significance from baseline, but was not statistically different compared to placebo.

On the ADAS-cog-11, the placebo arm showed a trend towards worsening (increase) (FIG. 3B). While the Lomecel-B arms appeared more stable, these were not significant from placebo.

There were no significant changes from baseline for any of the arms on the TMT-A, and no differences between the Lomecel-B arms and placebo (FIG. 3C). For the TMT-B, the placebo arm showed a trending worsening (longer completion time), whereas both Lomecel-B arms showed trending improvements that did not reach statistical significance (FIG. 3D).

On the GDS, no significant differences from baseline occurred in any of the arms, or in the Lomecel-B arms versus placebo (FIG. 3E).

### Quality-of-Life and Activities-of-Daily-Living Assessments:

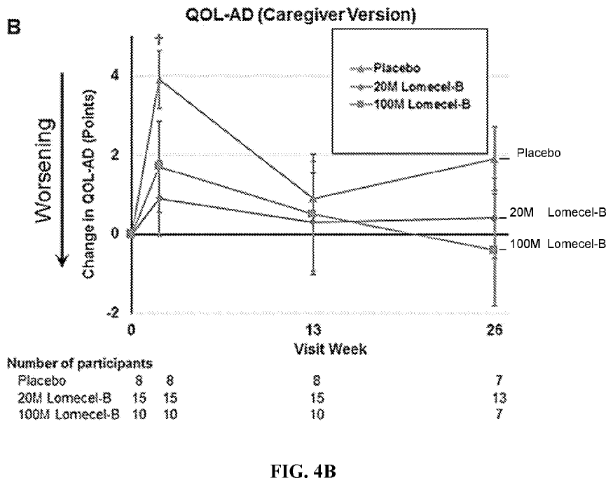

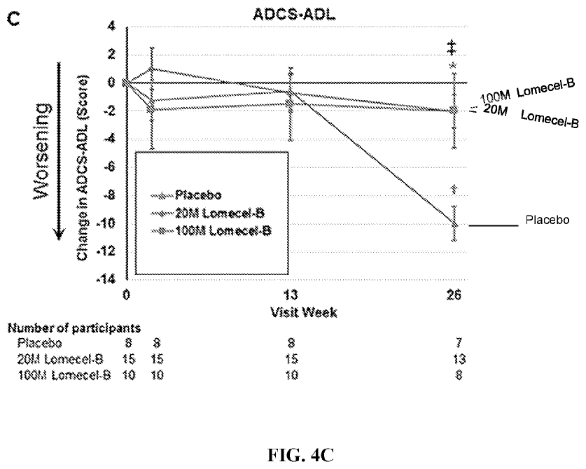

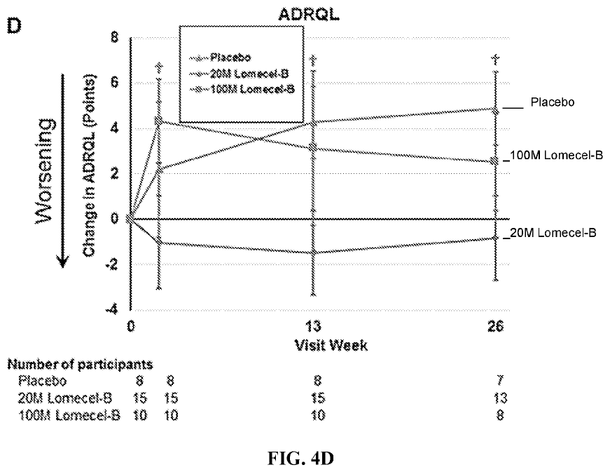

On the patient version of the QOL-AD, the 20M Lomecel-B arm showed significant improvement versus placebo at Week 26 by 3.85±1.943 points (p=0.0444; 2-sided 95% CI 0.13-9.12) (FIG. 4A). There was no significant difference between the 100M Lomecel-B and placebo arms, and there was no significant changes in these arms from baseline. The caregiver version of the QOL-AD showed trending improvements in all arms, in which the placebo arm showed a significant change from baseline at Week 2 (3.9±4.61 points; p=0.0491; 95% CI 0.02-7.73) (FIG. 4B). There were no significant changes from baseline in the Lomecel-B arms relative to the change in placebo.

On the ADCS-ADL, the placebo arm significantly declined (worsened) at Week 26 by 9.27±2.782 points (p=0.0211; 2-sided 95% CI −17.83-−2.10) (FIG. 4C). This change was significant relative to the 20M Lomecel-B arm by 6.95±3.46 points at Week 26 (p=0.0118; 95% CI 1.99-13.94). Similarly, this difference was also significant in the combined Lomecel-B arms versus the change in placebo at Week 26 of 6.96±3.125 point (p=0.0080; 2-sided 95% CI 2.26-13.67). Neither Lomecel-B arm showed a decline from baseline.

There was a significant improvement (increase) in the ADRQL from baseline in the 100M Lomecel-B arm at Week 2 (4.33±5.88 points; p=0.0449; 95% CI 0.12-8.54). The placebo arm also showed significant increases at Week 13 (2.21±8.44 points; p=0.0308; 95% CI 0.53-8.04) and Week 26 (4.28±4.49 points; p=0.0225; 95% CI 0.97-8.83) (FIG. 4D). The 20M Lomecel-B arm showed no significant change from baseline, and there were no significant changes in either Lomecel-B arm versus placebo.

### Serum-Based Biomarkers:

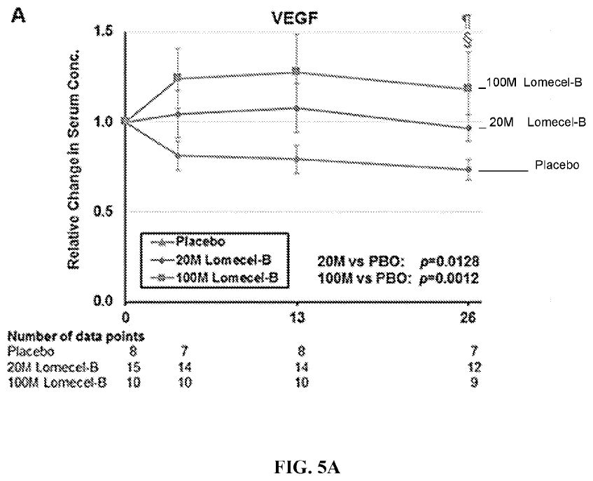

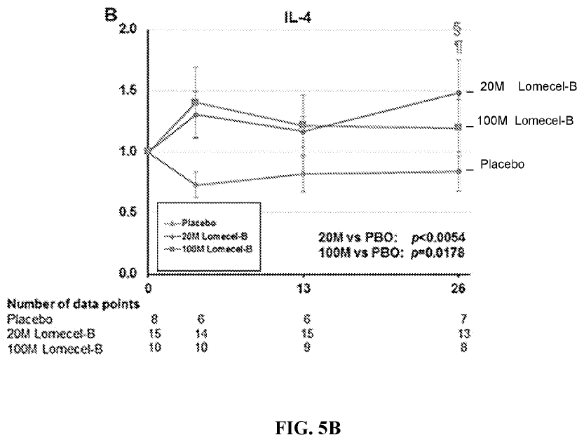

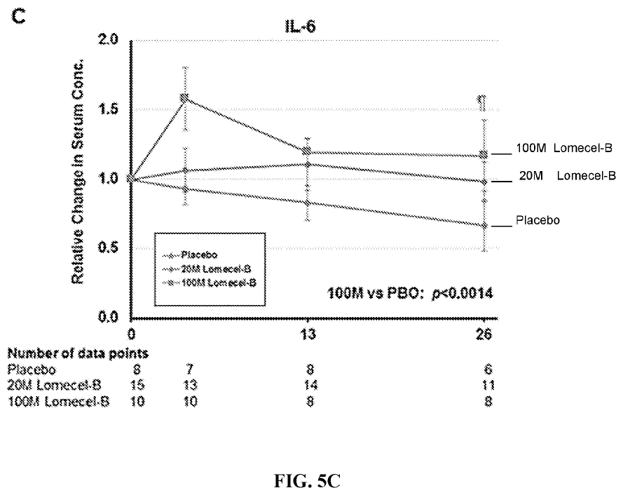

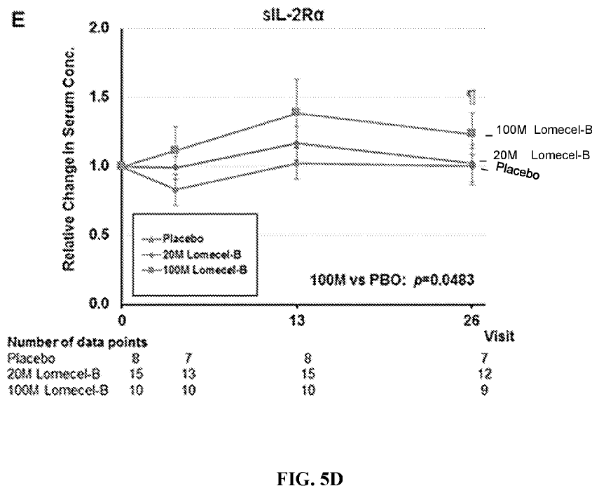

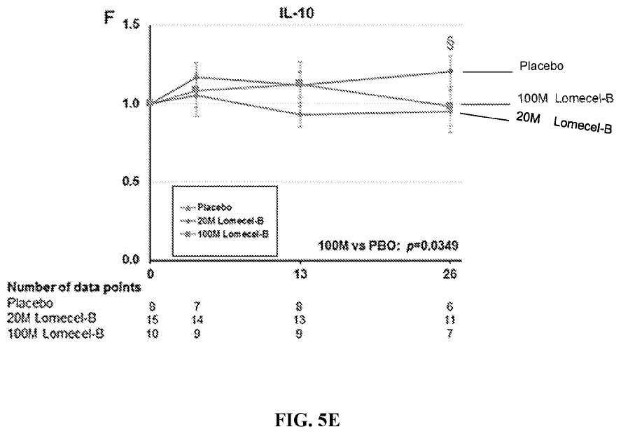

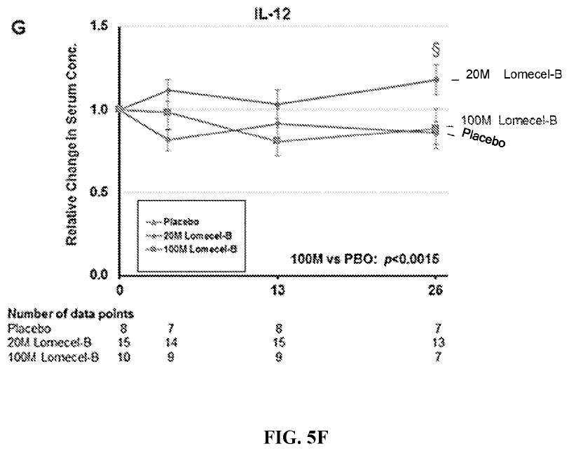

Post-treatment vascular-related biomarkers were significantly higher in the Lomecel-B arms versus placebo. For VEGF, the placebo arm showed a significant decrease through Week 26 versus both the 20M Lomecel-B (p<0.0128) and 100M Lomecel-B (p<0.0012) arms (FIG. 5A). Similarly, IL-4 significantly decreased in the placebo arm versus both the 20M (p<0.0054) and 100M Lomecel-B arms (p<0.0180) (FIG. 5B). IL-6 also significantly decreased in the placebo arm versus the 100M Lomecel-B (p<0.0014) (FIG. 5C). A significant increase in D-dimer was found in the 100M Lomecel-B arm versus placebo (FIG. 5D), but no significance was seen in the 20M Lomecel-B arm versus placebo.

Post-treatment anti-inflammatory biomarkers were significantly higher in the Lomecel-B arms versus placebo. sIL-2Rα significantly increased in the 100M Lomecel-B arm versus placebo (p<0.0049) (FIG. 5E). The 20M Lomecel-B arm had significantly increased IL-10 versus placebo (p<0.0349) (FIG. 5F) as well as IL-12 (p<0.0015) (FIG. 9E).

Serum levels of Aβ₃₈, Aβ₄₀, and Aβ₄₂ trended higher in the Lomecel-B arms versus placebo (Table 3).

TABLE 3
Changes in Neuronal-Related Serum Biomarkers

| Biomarker | Visit Week | | | | |
|---|---|---|---|---|---|
| Study arm | 0 | 4 | 13 | 26 | p-value * |

Aβ₃₈

| | | | | | |
|---|---|---|---|---|---|
| Placebo | 143.9 ± 228.7(8) | 100.2 ± 194.6(7) | 87.3 ± 171.6(8) | 88.1 ± 131.6(7) | |
| 20M Lomecel-B | 37573.2 ± 136662.1(15) | 44181.7 ± 156695.2(14) | 38526.3 ± 133230.4(15) | 45410.9 ± 154909.9(13) | 0.220 |
| 100M Lomecel-B | 41 ± 28.5(9) | 26.6 ± 0(10) | 62.2 ± 50.9(9) | 32.4 ± 16.5(8) | 0.192 |

Aβ₄₀

| | | | | | |
|---|---|---|---|---|---|
| Placebo | 65 ± 60.4(8) | 73.3 ± 45.7(7) | 47 ± 29(8) | 82.6 ± 78(7) | |
| 20M Lomscel-B | 2458 ± 7676(15) | 3266.2 ± 10837.8(14) | 1954.8 ± 8491.6(16) | 2767.6 ± 9143.7(13) | 0.895 |
| 100M Lomecel-B | 82.2 ± 47.8(9) | 71.9 ± 47(10) | 79.7 ± 49.2(9) | 70.5 ± 30(8) | 0.500 |

Aβ₄₂

| | | | | | |
|---|---|---|---|---|---|
| Placebo | 12.9 ± 11.2(8) | 11 ± 11.2(7) | 10.5 ± 11(8) | 14 ± 15.1(7) | |
| 20M Lomecel-B | 1061.4 ± 3520.2(18) | 1192.4 ± 3917(14) | 974.7 ± 3258.4(15) | 1214.4 ± 3871.9(13) | 0.781 |
| 100M Lomecel-B | 11.5 ± 7.2(9) | 13.5 ± 11.3(10) | 17.5 ± 14.1(9) | 14.6 ± 8.5(8) | 0.132 |

### Hippocampal Volumetry:

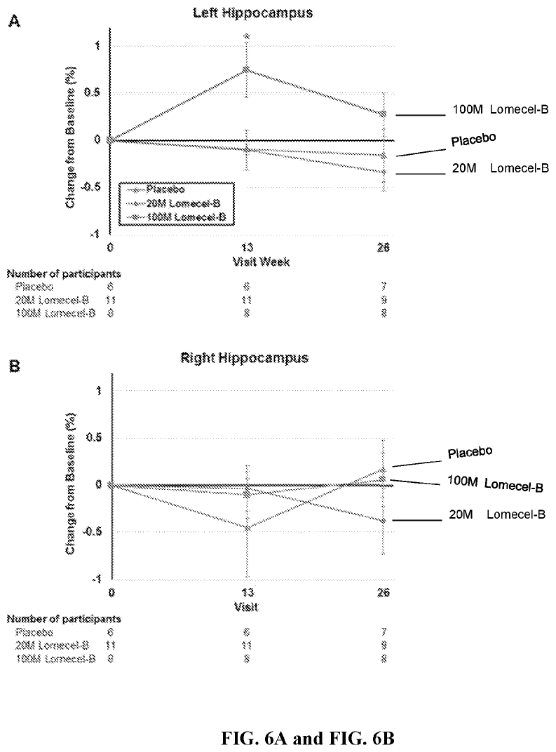

Brain volumetry revealed a significant increase in left hippocampal volume at Week 13 in the 100M Lomecel-B arm versus the change in placebo (p=0.0311) (FIG. 6A). By Week 26, this increase dropped and was no longer statistically significant versus placebo. The 20M Lomecel-B arm showed no significant difference versus placebo. In contrast, no significant changes were seen in the right hippocampus for either Lomecel-B versus placebo (FIG. 6B). For this analysis, hippocampal size was normalized to hippocampal fissure volume to correct for cranial size differences.

### Results:

The major new findings of this placebo-controlled trial are that an intravenous infusion of Lomecel-B in patients with mild AD is safe and well-tolerated, potentially improves neurocognition and quality of life in treated patients, and produces biologically plausible alterations in serum biomarkers. Additionally, this trial revealed important insights into cell dosing and duration of effect, suggesting that a low dose may be more effective than a higher dose. Together the results of this trial pave the way for future larger clinical trials powered to detect clinical efficacy endpoints.

This trial is supported by preclinical results, and is based upon solid pathophysiologic therapeutic rationale of addressing the neuroinflammatory and vascular impairment hypotheses of AD pathogenesis. Given the well-characterized anti-inflammatory and vascular effects of MSCs, we designed a placebo-controlled trial for evaluation in mild AD.

Several lines of evidence from pre-specified measures encourage Lomecel-B as a disease modifying intervention for AD via improvements in all efficacy domains studied: neurocognitive and neuropsychological; QOL and ADLs; and biomarkers. In the clinical effect domains, the 20M Lomecel-B arm showed significant benefits versus placebo on the MMSE, patient QOL-AD, and ADRQL, however this results are secondary outcome and must be taken cautiously. More importantly, neither of the Lomecel-B arms showed a significant worsening from baseline on any of the clinical assessments, which was not the case for placebo, and which further supports the safety of Lomecel-B.

With regard to biomarkers, we detected significant changes in circulating biomarkers in two categories: vascular-related (VEGF, IL-4, IL-6) and anti-inflammatory (IL-4, IL-10, IL-12, and sIL-2Rα). These changes were mostly dose-dependent, in which the placebo declined, the 100M dose showed the largest significant increase, and the 20M dose was in-between or similar to the 100.

Changes in the vascular-related biomarkers are consistent with neurovascular improvement. VEGF, which exhibited important alterations, has neuroprotective and neurorestorative effects, and positively associates with increased hippocampal volume, which was also observed in this study. IL-4 is a pleiotropic cytokine that regulates vascular function, cell proliferation and apoptosis, and decreases pro-inflammatory profiles of a variety of cell types, including microglia, and can induce BDNF production from astrocytes. IL-4 can also improve Aβ-inhibited long-term potentiation (LTP) by suppressing Aβ-induced upregulation of IL-1β from M1 microglial activation. IL-4 also leads to clearance of oligomeric Aβ peptides by increasing expression of the Aβ-degrading enzyme CD10 in microglia. Furthermore, IL-4 can activate a M2 microglia phenotype which in turn promotes neurogenesis and oligodendrogenesis, and positively correlates with left subiculum volume in patients with mild cognitive impairment. In vivo injection of IL-4 in the APP23 AD mouse model reduced Aβ levels and significantly improved memory deficits. IL-6 is also a pleiotropic cytokine that can have beneficial effect, such as under exercise conditions, has pro-angiogenic-osteogenic activity, and can protect from glucose toxicity via VEGF signaling.

The anti-inflammatory biomarker increases in the Lomecel-B arms are consistent with decreased systemic inflammation and neuroinflammation. Since neuroinflammation appears requisite for the manifestation of dementia, the increased anti-inflammatory cytokine profile is consistent with the clinical assessment improvements. IL-10 has well-documented anti-inflammatory properties. IL-12 has anti-inflammatory and pro-inflammatory activities that are contextual dependent, and induces IL-10 expression as part of its anti-inflammatory roles. In the context of AD, IL-12 is markedly lower in the CSF of AD patients compared to normal subjects, and both IL-10 and IL-12 were increased after Lomecel-B treatment in this study. In culmination with the anti-inflammatory roles of sIL-2Rα and IL-4, these results suggest anti-inflammatory synergy in response to Lomecel-B treatment.

Circulating levels of Aβ peptides showed trending higher levels in the Lomecel-B arms versus placebo. Plasma Aβ₄₂ is moderate decreased in in preclinical/prodromal AD stages, and Aβ₄₀ and Aβ₄₂ show even greater significant decreases in AD. The Aβ trends seen in the Lomecel-B arms would be consistent with improved cognitive status seen in the patients.

Finally, adult neurogenesis significantly declines in AD. The increase in hippocampal volume would be consistent with an increase in neurogenesis in these patients, and the improvements in the other efficacy domains.

### REFERENCES

1. Sheppard O, Coleman M. Alzheimer's Disease: Etiology, Neuropathology and Pathogenesis. In: Huang X, editor. Alzheimer's Disease: Drug Discovery. Brisbane (AU)2020. p. 6.
2. Lue L F, Brachova L, Civin W H, Rogers J. Inflammation, A beta deposition, and neurofibrillary tangle formation as correlates of Alzheimer's disease neurodegeneration. J Neuropathol Exp Neurol. 1996; 55(10):1083-8. Epub 1996/10/01. PubMed PMID: 8858005.
3. Pimplikar S W. Neuroinflammation in Alzheimer's disease: from pathogenesis to a therapeutic target. J Clin Immunol. 2014; 34 Suppl 1:S64-9. Epub 2014/04/09. doi: 10.1007/s10875-014-0032-5. PubMed PMID: 24711006.
4. Heneka M T, Carson M J, El Khoury J, Landreth G E, Brosseron F, Feinstein D L, et al. Neuroinflammation in Alzheimer's disease. Lancet Neurol. 2015; 14(4):388-405. Epub 2015/03/21. doi: 10.1016/S1474-4422(15)70016-5. PubMed PMID: 25792098.
5. Monson N L, Ireland S J, Ligocki A J, Chen D, Rounds W H, Li M, et al. Elevated CNS inflammation in patients with preclinical Alzheimer's disease. J Cereb Blood Flow Metab. 2014; 34(1):30-3. doi: 10.1038/jcbfm.2013.183. PubMed PMID: 24149932; PubMed Central PMCID: PMC3887357.
6. Steinman J, Sun H S, Feng Z P. Microvascular Alterations in Alzheimer's Disease. Front Cell Neurosci. 2020; 14:618986. doi: 10.3389/fncel.2020.618986. PubMed PMID: 33536876; PubMed Central PMCID: PMCPMC7849053.
7. Marques F, Sousa J C, Sousa N, Palha J A. Blood-brain-barriers in aging and in Alzheimer's disease. Mol Neurodegener. 2013; 8:38. doi: 10.1186/1750-1326-8-38. PubMed PMID: 24148264; PubMed Central PMCID: PMCPMC4015275.
8. Zhao Z, Nelson A R, Betsholtz C, Zlokovic B V. Establishment and Dysfunction of the Blood-Brain Barrier. Cell. 2015; 163(5):1064-78. doi: 10.1016/j.cell.2015.10.067. PubMed PMID: 26590417; PubMed Central PMCID: PMCPMC4655822.
9. Tarasoff-Conway J M, Carare R O, Osorio R S, Glodzik L, Butler T, Fieremans E, et al. Clearance systems in the brain-implications for Alzheimer disease. Nat Rev Neurol. 2015; 11(8):457-70. doi: 10.1038/nmeurol.2015.119. PubMed PMID: 26195256; PubMed Central PMCID: PMCPMC4694579.
10. Erickson M A, Banks W A. Blood-brain barrier dysfunction as a cause and consequence of Alzheimer's disease. J Cereb Blood Flow Metab. 2013; 33(10):1500-13. doi: 10.1038/jcbfm.2013.135. PubMed PMID: 23921899; PubMed Central PMCID: PMCPMC3790938.
11. Hayashi S, Sato N, Yamamoto A, Ikegame Y, Nakashima S, Ogihara T, et al. Alzheimer disease-associated peptide, amyloid beta40, inhibits vascular regeneration with induction of endothelial autophagy. Arterioscler Thromb Vasc Biol. 2009; 29(11):1909-15. Epub 2009/10/10. doi: 10.1161/ATVBAHA.109.188516. PubMed PMID: 19815818.
12. Klohs J. An Integrated View on Vascular Dysfunction in Alzheimer's Disease. Neurodegener Dis. 2019; 19 (3-4):109-27. doi: 10.1159/000505625. PubMed PMID: 32062666.
13. Oliva A A, McClain-Moss L, Pena A, Drouillard A, Hare J M. Allogeneic mesenchymal stem cell therapy: A regenerative medicine approach to geroscience. Aging Med (Milton). 2019; 2(3):142-6. doi: 10.1002/agm2.12079. PubMed PMID: 31667462; PubMed Central PMCID: PMCPMC6820701.
14. Pittenger M F, Discher D E, Peault B M, Phinney D G, Hare J M, Caplan A I. Mesenchymal stem cell perspective: cell biology to clinical progress. NPJ Regen Med. 2019; 4:22. doi: 10.1038/s41536-019-0083-6. PubMed PMID: 31815001; PubMed Central PMCID: PMCPMC6889290.
15. Eggenhofer E, Luk F, Dahlke M H, Hoogduijn M J. The life and fate of mesenchymal stem cells. Front Immunol. 2014; 5:148. doi: 10.3389/fimmu.2014.00148. PubMed PMID: 24904568; PubMed Central PMCID: PMC4032901.
16. Lalu M M, McIntyre L, Pugliese C, Fergusson D, Winston B W, Marshall J C, et al. Safety of cell therapy with mesenchymal stromal cells (SafeCell): a systematic review and meta-analysis of clinical trials. PloS one. 2012; 7(10):e47559. doi: 10.1371/journal.pone.0047559. PubMed PMID: 23133515; PubMed Central PMCID: PMCPMC3485008.
17. Karantalis V, Schulman I H, Balkan W, Hare J M. Allogeneic cell therapy: a new paradigm in therapeutics. Circ Res. 2015; 116(1):12-5. doi: 10.1161/CIRCRESAHA.114.305495. PubMed PMID: 25552688; PubMed Central PMCID: PMCPMC4411634.
18. Neves A F, Camargo C, Premer C, Hare J M, Baumel B S, Pinto M. Intravenous administration of mesenchymal stem cells reduces Tau phosphorylation and inflammation in the 3×Tg-AD mouse model of Alzheimer's disease. Experimental neurology. 2021:113706. doi: 10.1016/j.expneurol.2021.113706. PubMed PMID: 33757765.
19. Kim S, Chang K A, Kim J, Park H G, Ra J C, Kim H S, et al. The preventive and therapeutic effects of intravenous human adipose-derived stem cells in Alzheimer's disease mice. PloS one. 2012; 7(9):e45757. doi: 10.1371/joumal.pone.0045757. PubMed PMID: 23049854; PubMed Central PMCID: PMC3458942.
20. Shin J Y, Park H J, Kim H N, Oh S H, Bae J S, Ha H J, et al. Mesenchymal stem cells enhance autophagy and increase beta-amyloid clearance in Alzheimer disease models. Autophagy. 2014; 10(1):32-44. doi: 10.4161/auto.26508. PubMed PMID: 24149893.
21. Lee J K, Schuchman E H, Jin H K, Bae J S. Soluble CCLS derived from bone marrow-derived mesenchymal stem cells and activated by amyloid beta ameliorates Alzheimer's disease in mice by recruiting bone marrow-induced microglia immune responses. Stem Cells. 2012; 30(7):1544-55. doi: 10.1002/stem.1125. PubMed PMID: 22570192.
22. Bae J S, Jin H K, Lee J K, Richardson J C, Carter J E. Bone marrow-derived mesenchymal stem cells contribute to the reduction of amyloid-beta deposits and the improvement of synaptic transmission in a mouse model of pre-dementia Alzheimer's disease. Curr Alzheimer Res. 2013; 10(5):524-31. doi: 10.2174/15672050113109990027. PubMed PMID: 23036020.

1. A method for alleviating the symptoms of Alzheimer's disease (AD) in a subject in need thereof, wherein the method comprises administering a composition comprising a therapeutically effective amount of allogeneic mesenchymal stem cells (MSCs) to the subject.

2. A method for treating Alzheimer's disease (AD) or inhibiting AD disease progression, wherein the method comprises administering a composition comprising a therapeutically effective amount of allogeneic mesenchymal stem cells (MSCs) to the subject.

3. The method according to claim 1, wherein the method further comprises measuring the concentration of a biomarker or biomarkers in the subject suffering from symptoms of AD before and after the administration of the composition comprising allogenic MSCs.

4. The method according to claim 1, wherein the method further comprises measuring the cognitive function of the subject suffering from symptoms of AD before and after administration of the composition comprising allogenic MSCs.

5. The method according to claim 3, wherein the biomarkers comprise cytokines selected from the group consisting of IL-4, IL-6, IL-8, IL-10, IL-12p70, IL-17, sIL-2Rα or combinations thereof.

6. The method according to claim 5, wherein the cytokine's concentration is increased in the serum, plasma, cerebral spinal fluid or blood of the subject in need thereof after administration of the composition comprising a therapeutically effective amount allogenic MSCs.

7. The method according to claim 6, wherein the cytokine concentration increase is from 0.5% to 10%, 5% to 10%, 10% to 50%, or greater than 50%.

8. The method according to claim 3, wherein the biomarkers further comprise neuronal-related molecules or peptides selected from the group consisting of tau, phospho-tau, Aβ-38, Aβ-40, Aβ-42, NFL or combinations thereof.

9. The method according to claim 8, wherein the concentration of Aβ-38, Aβ-40 or Aβ-42 is increased in the serum, plasma, or blood of the subject in need thereof after administration of the composition comprising a therapeutically effective amount allogenic MSCs.

10. The method according to claim 9, wherein the concentration of Aβ-38, Aβ-40 or Aβ-42 is increased from 0.5% to 10%, 5% to 10%, 10% to 50%, or greater than 50%.

11. The method according to claim 8, wherein the concentration of tau, phospho-tau or NFL is decreased in the serum, plasma, cerebral spinal fluid or blood of the subject in need thereof after administration of the composition comprising a therapeutically effective amount allogenic MSCs.

12. The method according to claim 11, wherein the concentration of tau, phospho-tau or NFL is decreased from 0.5% to 10%, 5% to 10%, 10% to 50%, or greater than 50%.

13. The method according to claim 3, wherein the biomarkers further comprise inflammation signaling molecules such as pro-BNP, TNF-α, or combinations thereof.

14. The method according to claim 13, wherein the concentration pro-BNP or TNF-α is decreased in the serum, plasma, cerebral spinal fluid or blood of the subject in need thereof after administration of the composition comprising a therapeutically effective amount allogenic MSCs.

15. The method according to claim 14, wherein the concentration of pro-BNP or TNF-α is decreased from 0.5% to 10%, 5% to 10%, 10% to 50%, or greater than 50%.

16. The method according to claim 3, wherein the biomarkers further comprise VEGF.

17. The method according to claim 16, wherein the concentration of VEGF is increased in the serum, plasma, cerebral spinal fluid or blood of the subject in need thereof after administration of the composition comprising a therapeutically effective amount allogenic MSCs.

18. The method according to claim 17, wherein the concentration of VEGF is decreased from 0.5% to 10%, 5% to 10%, 10% to 50%, or greater than 50%.

19. The method according to claim 3, wherein the method further comprises determining the change in the size of areas in the subject's brain after administration of the composition comprising allogeneic MSCs.

20. The method according to claim 19, wherein the areas in the subject's brain that change in size after administration of the composition are selected from the group consisting of the amygdala, cortical nucleus, the hippocampus, hippocampal subregions, and/or the corticoamygdaloid transition.

21. The method according to claim 3, wherein the method further comprises determining if a change occurs in the corticoamygdaloid transition of the subject after administration of the composition comprising allogeneic HMCs.

22. The method according to claim 3, wherein the composition comprises 20×10⁶ MSCs.

23. The method according to claim 3, wherein the composition comprises 100×10⁶ MSCs.

24. The method according to claim 3, wherein the method further comprises examining the cerebral spinal fluid of the subject before and after administration of the composition comprising allogeneic HMCs.
